(19) United States
(12) Patent Application Publication
(10) Pub. No.: US 2003/0082803 A1
(43) Pub. Date: May 1, 2003

(54) Cell cycle control

(76) Inventors: Stephen Dalton, Westbourne Park (AU); Elaine Marie Stead, Ridgehaven (AU)

Correspondence Address: Sutherland Asbill & Brennan, LLP, 999 Peachtree St. N.E, Atlanta, GA, 30309, US

(21) Appl. No.: 10/274,585

(22) Filed: Oct. 21, 2002

Related U.S. Application Data
(63) Continuation-in-part of application No. 10/089,130.
Application No. 10/089,130 is a 371 of international application No. PCT/AU00/01184, filed on Sep. 22, 2000.

(30) Foreign Application Priority Data
Sep. 24, 1999 (AU) PQ3073

(51) Int. Cl. A01K 67/00; A61K 48/00; C12N 5/06; C12N 5/08; A61K 31/365

(52) U.S. Cl. 435/368; 800/8; 435/325; 424/93.21; 514/473

(57) ABSTRACT

A method of identifying and/or selecting non-differentiating pluripotent or pluripotent-related cells from a mixed cell population including differentiating and non-differentiating pluripotent or pluripotent-related cells, which method includes reducing kinase activity and/or expression in said cells.

The present invention relates to methods for using molecules relating to the control of the cell cycle, and cell proliferation, to improve technology relating to pluripotent, multipotent and differentiated cells. More particularly, the present invention relates to methods for identifying pluripotent cells and partially differentiated cells, to methods for enhancing the maintenance and proliferation of pluripotent cells and partially differentiated cells, to methods for isolating new populations of pluripotent cells and partially differentiated cells, and facilitating their maintenance and proliferation in vitro, to methods for reprogramming of differentiated somatic cells so that the cells are converted to a less differentiated state, including to a state of pluripotency or multipotency, and to methods for selecting undifferentiated cells, in a mixed population of cells comprised of differentiated and undifferentiated cells.

This invention also relates to methods for regulating the differentiation of cells, including pluripotent cells and multipotent cells, and to methods for prolonging the lifespan in vitro of pluripotent or multipotent cells. Also within the scope of the present invention are cells, embryos and animals produced using the methods referred to above.

In this patent application the term “pluripotent” refers to cells that can contribute substantially to all tissues of the developing embryo. “Multipotent” or “partially differentiated” refers to partially differentiated cells that are able to differentiate further into more than one terminally differentiated cell type. Such cells include, but are not limited to haematopoietic stem cells and neural stem cells.

“Maintenance of pluripotent cells” is to be understood as the maintenance of such cells in vitro in an undifferentiated state. It may also include, but does not always include, the understanding that these cells are immortal.

The cell division cycle is normally composed of four distinct phases, which in typical somatic cells take 18-24 hours to complete. The S-phase represents the period when chromosomal DNA is duplicated, this is then followed by a gap phase (G2) where cells prepare to segregate chromosomes between daughter cells during M-phase. After completion of M-phase, cells enter a second gap phase, G1, which separates M- from S-phase. G1 is of special significance because it is here that a cell decides to continue dividing or withdraw from the cell cycle.

At the molecular level, the cell cycle is controlled by waves of cyclin-dependent protein kinase (Cdk) activities that are activated only at specific times and which drive the cell cycle transitions by phosphorylation of specific substrates. For activity, each Cdk catalytic subunit requires a cyclin regulatory subunit. Cdks acting at the G1 phase include Cdk2 which is regulated by cyclin E, and Cdk4 and Cdk6 which are regulated by cyclin D activities. Additional levels of control are provided by cyclin-dependent kinase inhibitors, such as p16.

The availability of murine pluripotent cells in vitro has led to the development of powerful model systems for investigating mechanisms of early development. In particular pluripotent cells provide an opportunity to investigate the molecular events responsible for the close association between pluripotency and cell cycle characteristics, and the link between differentiation and changes in regulation of the cell cycle.

Murine pluripotent cells can be isolated from the preimplantation embryo and maintained in vitro as ES cells. ES cells retain pluripotence indefinitely and display the properties of stem cells, including competency to differentiate into all cell types, and the ability for indefinite self-renewal. Early primitive ectoderm-like (EPL) cells are also pluripotent stem cells. They differ in some properties to ES cells, and have the capacity to revert to ES cells in vitro. They can be derived from ES cells or other types of pluripotent cells, and are the in vitro equivalent of primitive ectoderm cells of postimplantation embryos. As such, EPL cells can also be established in vitro from cells isolated from the primitive ectoderm of postimplantation embryos. The properties of EPL cells, factors required for their maintenance and proliferation in vitro, and their ability to differentiate uniformly in vitro to form essentially homogeneous populations of partially differentiated and differentiated cell types are described fully in PCT/AU99/00265, to applicants, the entire disclosure of which is incorporated herein by reference. Cells of the primordial gonad, primordial germ cells (PGCs), also retain pluripotency during embryonic development, and can be isolated and cultured in vitro as embryonic gonadal (EG) cells. Embryonic carcinoma (EC) cells may also be pluripotent.

While pluripotent cells and partially differentiated cells have long been recognised as ideally suited to a range of applications, in practice technical barriers have generally restricted their use in the prior art.

Significant problems have been encountered in the realisation of this technology. In many cases, identification and culture of stem cells for target tissues has not been achieved. Furthermore, even when cell populations can be enriched, for example in the case of haemopoietic stem cells, these have proven refractory to genetic manipulation. In particular the failure of cultured stem cells to proliferate clonally in culture prevents the use of homologous recombination-based techniques for modification of endogenous genes.

It is clear that there have been major difficulties in the successful isolation, maintenance in vitro, genetic manipulation and germ-line transmission of pluripotent cells from species other than mouse. These difficulties have severely restricted the application of these technologies for commercial, medical and agricultural benefit.

There have also been difficulties that have restricted the successful reprogramming of differentiated cells so that they revert to a pluripotent, or less differentiated state. In particular there have been difficulties in trapping cells in a pluripotent or partially differentiated state following spontaneous dedifferentiation, or dedifferentiation occurring as a result of an environment or factors that promote dedifferentiation. Similarly approaches for the selection of pluripotent cells or partially differentiated cells from cell populations comprised of pluripotent cells, partially differentiated cells and differentiated cells have been limited. Nuclear transfer has been one of the approaches suggested in the prior art to achieve reprogramming of differentiated somatic cells. However nuclear transfer approaches have been inefficient due at least in part, to difficulties in reprogramming. Furthermore there are major ethical problems with the use of human oocytes in reprogramming human somatic cells.

There have also been problems in controlling the differentiation of pluripotent cells along defined differentiation pathways.

There have also been difficulties associated with primary somatic cells in vitro. In particular it has been difficult to maintain primary cells in culture for prolonged periods, due to biological mechanisms that limit the number of proliferation rounds that such cells can undergo. The consequence of this limitation, termed the Hayflich limit, is that the ability to genetically modify primary or untransformed cells in vitro has been restricted. This limitation has restricted the utility of these cells for commercial, agricultural or medical benefit.

In International patent application PCT/AU00/01184 to applicants, the unusual mode of cell cycle regulation seen in pluripotent cells is discussed. This new understanding has permitted advances in cell cycle control. However, difficulties remain, particularly in controlling a cell cycle which is at times very rapid.

It is accordingly an object of the present invention to overcome or at least alleviate one or more of the difficulties or deficiencies associated with the prior art.

Accordingly, in a first aspect of the present invention there is provided a method for identifying and/or selecting non-differentiating pluripotent or pluripotent-related cells from a mixed cell population including differentiating and non-differentiating pluripotent or pluripotent-related cells, which method includes reducing kinase activity and/or expression in said cells.

Applicants have discovered that pluripotent cells and pluripotent-related cells, e.g. of embryonic origin, proliferate at unusually rapid rates and that such rapid cell divisions are driven by extreme levels of kinase (e.g. Cdk2) activity.

Applicants have surprisingly found that reducing kinase activity will slow the cell cycle of pluripotent cells but without otherwise substantially affecting the cell cycle structure, but will arrest the mitotic and/or physiological activity of differentiating or differentiated cells. Accordingly by reducing kinase activity a method is provided for preferentially selecting pluripotent cells from a mixed population of differentiated, partially differentiated and pluripotent cells. Further, a method is provided for preferentially selecting robust healthy pluripotent cells from less robust pluripotent cells.

In a preferred aspect the identification method may include reducing the expression and/or activity of a cyclin-dependent protein kinase, e.g. (Cdk2) in said cells.

The kinase activity may be reduced by introducing a kinase inhibitor. A chemical inhibitor is preferred. The chemical inhibitor may be selected from one or more of the group consisting of Ro09-3033, roscovitine, olomoucine, butyrolactone and flavopiridol. The inhibitor Ro09-3033 is preferred.

The pluripotent cells may be selected from embryonic stem (ES) cells, early primitive ectoderm-like (EPL) cells, and other pluripotent cells of embryonic origin. The pluripotent cells may be murine or other mammalian, including human, origin. Applicants have established that human ES cells have a similar all cycle structure to murine pluripotent cells.

In a preferred embodiment of the present invention, applicants have surprisingly found that treatment of a mixed cell population may selectively arrest a proportion of non-differentiating pluripotent or pluripotent-related cells exhibiting reduced viability in vitro. Accordingly, the method according to this aspect of the present invention may further include

selectively arresting the cell cycle of non-differentiating pluripotent or pluripotent-related cells exhibiting reduced viability in vitro.

Accordingly, the method of selection according to the embodiment of the present invention permits selection not only of pluripotent cells from differentiating cells but also an improvement in cell quality or viability.

In a further aspect of the present invention there is provided a method for modifying the cell cycle of a non-differentiating pluripotent or pluripotent-related cell, which method includes reducing kinase activity and/or expression in said cell.

As stated above, applicants have surprisingly discovered that it is possible to slow the rate of the cell cycle of pluripotent cells, e.g. of embryonic origin without substantially effecting the cell cycle structure. For example, for murine ES cells, the generation times for untreated cells was approximately 11 hours and for inhibition-treated cells, approximately 18.3 hours.

Preferably the pluripotent or pluripotent-related cells are identified by the presence of one or more of the following characteristics:

a pluripotent-specific cell cycle exhibiting a rapid cycle with short gap phases;

elevated constitutive expression and/or activity of cyclin E;

elevated constitutive expression and/or activity of cyclin A;

pluripotent-specific expression of Cdk inhibitors; and

presence of a phosphorylated tumour suppressor protein.

In a further embodiment of this aspect of the present invention, the pluripotent-related cells may include multipotent cells (such as haemopoietic stem cells and neural stem cells). The multipotent cells may be derived by partial differentiation of pluripotent cells and which are capable of differentiating further into a number of different cell types, which may have all or some of the above cell cycle features.

In a further aspect, the present invention provides a method of regulating the mitotic and/or physiological activities, and differentiation potential of a pluripotent or pluripotent-related cell, which method includes reducing kinase activity and/or expression in said cells.

As discussed above, the duration of the cell cycle for pluripotent, e.g. embryonic cells may be significantly extended by reducing kinase (e.g. Cdk2) activity.

The method of regulation described above may be applied to facilitate maintenance and/or promote proliferation to enhance pluripotent or multipotent in vitro.

As stated above, the pluripotent cells may be of any suitable type and may be in vitro or in vivo. Preferably, the pluripotent cells are selected from one or more of the group consisting of epiblast cells, ES cells, EPL cells (as described in International Patent application PCT/AU99/0265), primordial germ cells (PGCs) and embryonic carcinoma (EC) cells.

Similarly the multipotent cells may be of any suitable type and may be in vitro or in vivo. They may be any partially differentiated cell type, including such cells as haematopoietic stem cells and neural stem cells.

The present invention will now be more fully described with reference to the accompanying figures and examples. It should be understood, however, that the description following is illustrative only and should not be taken in any way as a restriction on the generality of the invention described above.

### IN THE FIGURES

Pluripotent primitive ectoderm was surgically dissected from the epiblasts of thirty 6.25dpc mouse embryos, stained with propidium iodide and analysed for DNA content by flow cytometry. A whole mount in situ hybridisation of a representative 6.25dpc embryo probed with an antisense Oct4 RNA probe is shown, indicating the pluripotent (Oct4+) primitive ectoderm of the epiblast and the unstained extraembryonic region. Comparisons between cell cycle profiles of primitive ectoderm, mES cells, NIH 3T3 fibroblasts and MEFs are shown. The percentage of cells in G1, S and G2/M and average cell cycle lengths (hrs) are indicated.

FIG. 1

Embryonically-derived pluripotent cells have an unusual cell cycle structure and precocious Cdk activity.

(A) Morphology of murine D3 ES cell colonies (left panel) and D3-derived mEPL cells formed after passage for either two (mEPL-d2; middle panel) or four days (mEPL-d4; right panel) in HepG2-conditioned media. (B) Changes in marker gene expression associated with the ES to EPL transition. Total cell RNA (15 μg) was resolved on agarose-formaldehyde gels, blotted and probed with Rex-1, Fgf-5, Oct4 and GAPDH-specific probes. Transcript levels were quantitated by phosphorimaging and shown relative to levels of the GAPDH control. (C) mES, mEPL-d2, mEPL-d4 cells and MEFs were fixed, stained with propidium iodide and DNA content analysed by flow cytometry analysis. (D) Precocious histone H1 kinase activity associated with mES and mEPL cells. Histone H1 kinase activity was assayed in cyclin A, B, E and Cdk2 immunoprecipates from mES, mEPL-d2, mEPL-d4 and MEF whole cell lysates (100 μprotein). Levels of cyclin A, cyclin B, cyclin E and Cdk2 were evaluated by immunoblot analysis of extracts (15 μg protein) prepared from mES, mEPL-d2 and mEPL-d4 cells, Lower panel; quantitation of histone H1 kinase activity from immunoprecipitates. (E) Human ES cells have a similar cell cycle structure to murine pluripotent cells. Human ES cells (BresaGen Inc) were stained with propidium iodide and analysed for DNA content by flow cytometry.

FIG. 2

Histone H1 kinase activity associated with Cdk2, cyclin A and cyclin E is cell cycle independent. (A) Cdk2, cyclin A, cyclin E and cyclin B were immunoprecipitated from statically-blocked mES, mEPL (day 4) lysates (100 μg protein) or NIH 3T3 (400 μg protein) cell lysates and assayed for histone H1 kinase activity. Levels of input protein in each immunoprecipitation were shown to be equivalent by immunoblot analysis (15 μg input protein) of Cdk2 in each extract. Flow cytometry profiles of PI-stained cells are shown (bottom panel). Quantitation of histone H1 kinase activity is shown. (B) Synchronization of mES cells. mES cells were blocked and released as a synchronous population from a G1-S arrest into S-phase. Cells were fixed, stained with PI and analysed for cell cycle position based on DNA content by flow cytometry at hourly intervals over a 14 hour period. The front profile represents the nocodazole-blocked population (Noc) and the second (T=0) shows a G1-S population of cells released from this block into aphidicolin. Subsequent profiles represent the hourly intervals at which cells were sampled after release. (C) Immunoblot analysis of cyclin B, cyclin A, cyclin E and Cdk2 in extracts (15 μg protein) from the synchronization shown in (B). (D) Histone H1 kinase assays from Cdk2, cyclin A, cyclin E and cyclin B immunoprecipitates (100 μg protein). As a loading control (input protein) cell lysates (15 μg protein) used for immunoprecipitation were analysed by immunoblotting and probed with polyclonal antibodies raised against Cdk2 and cyclin A. (E) Quantitative analysis of histone H1 kinase activities from assays performed in (D). Relative levels of kinase activity were calculated after background subtraction from each lane and represent the average of two independent experiments.

FIG. 3

Ro09-3033 imposes a cell cycle arrest in Saos2 cells. (A) Structure of the Cdk2 inhibitor Ro09-3033. (B) Saos2 cells were grown in the presence of DMSO (0.5%), 0.1% FCS for 48 hours or 10 μM Ro09-3033 for 48 hours. Flow cytometry profiles of PI-stained cells are shown together with the percentage of cells in G₀/G1, S or G2/M.

FIG. 4

Cdk2 activity levels determine the rate of mES cell division but not cell cycle structure. (A) mES cells were grown for up to 8 passages in the presence (+) or absence (−) of Ro09-3033 (2 μM). Cdk2, cyclin E and cyclin B-associated H1 kinase activities were assayed as described previously after immunoprecipitation from whole cell lysates (100 μg protein). Input loading control was determined by probing immunoblots (15 μg lysate protein) with α-Cdk2 and α-Oct4 polyclonal antibodies. Quantitation of H1 kinase activities are shown in the lower panel as the average of two independent experiments. (B) Northern blot analysis of Oct4, Rex-1 and GAPDH transcripts from mES cells grown with (+) or without (−) inhibitor (2 μM) for the number of passages indicated. RNA was resolved by agarose gel electrophoresis, blotted and hybridized with [³²P]-labelled Oct4, Rex-1 and GAPDH probes (15 μg total RNA per lane). Lanes designated +/− were RNA samples from mES cells grown for either 2 or 12 passages with inhibitor followed by 4 passages in the absence of inhibitor. (C) After 8 passages in the presence (+) or absence (−) of 2 μM Ro09-3033, mES and mEPL cells were fixed and stained with PI and analysed by flow cytometry. The mean generation time (two independent experiments) for mES cells over this period is indicated in addition to the % of cells in G1, S and G2/M phases. Generation times of mEPL cells were not determined in this experiment.

### EXAMPLES

### Example 1

Materials and Methods

Cell Culture, Synchronization, Embryo Dissection and Flow Cytometry.

D3 mES cells (1) were cultured in the absence of feeders on tissue culture grade plastic-ware pre-coated with 0.1% gelatin-phosphate buffered saline (PBS), as described previously (2). ES cell culture medium consisted of Dulbecco's Modified Eagle Medium (DMEM, Gibco BRL) supplemented with 10% foetal calf serum (FCS, Commonwealth Serum Laboratories), 1 mM L-glutamine, 0.1 mM 2-mercaptoethanol, 100 U/ml penicillin, 100 U/ml streptomycin and 1000 U/ml recombinant human LIF (ESGRO) at 37° C. under 10% CO₂. mEPL cells were formed and maintained by culturing as described previously (2) in ES-media containing 50% conditioned media (MedII) supplemented with 1 mM L-glutamine, 0.1 mM 2-mercaptoethanol, 100 U/ml penicillin, 100 U/ml streptomycin and 1,000 U/ml recombinant LIF at a density of 2×10⁴ cells/cm². mEPL cells were passaged every 48 hours. MedII conditioned media was prepared from HepG2 cells (ATCC HB-8065) grown for 5 days in DMEM supplemented with 10% FCS, 40 mg/ml gentamycin and 1 mM L-glutamine, after being seeded at 5×10⁴ cells/ml. Cell supernatants were collected, filter sterilized then stored at 4° C. for up to 2 weeks. NIH 3T3 mouse fibroblast and early passage (passage 2-3) mouse embryonic fibroblasts (MEFs), prepared from outcrossed Swiss mice (3), were cultured in DMEM supplemented with 10% foetal calf serum, 1 mM L-glutamine, 100 U/ml streptomycin and 100 U/ml penicillin.

mES and mEPL cells were statically synchronised in prophase/mitosis by the addition of 45 ng/ml nocodazole (Sigma) to media for 12 hours or, in G1-S/S-phase by the addition of aphidicolin (Sigma) at 20 μg/ml for 12 hours. NIH 3T3 fibroblasts and MEFs were blocked by the addition of nocodazole and aphidicolin for 24 hours at the same concentrations used for mES and mEPL cells. For synchronization experiments, cells were blocked with nocodazole at 45 ng/ml for 12 hours after being plated for 48 hours, washed four times in PBS and released into fresh pre-warmed media supplemented with 20 μg/ml aphidicolin for 10 hours. The washing procedure was finally repeated allowing cells to be released into S-phase as a synchronous population. Cells were harvested from petri dishes, after a five-minute incubation in TEN buffer (40 mM Tris pH 7.4, 1 mM EDTA and 150 mM NaCl), by scraping with a rubber policeman followed by two washes in PBS.

Time-mated 6.25dpc Swiss mouse embryos were dissected by first prising open the mesometrial side of the decidua allowing the ‘shelled’ embryo to be released. After surgical removal of Reichardt's membrane and the extraembryonic region, visceral endoderm was peeled away from the epiblast by gently pipetting through a glass needle. Cells from pooled epiblasts were made into a single cell suspension by gentle pipetting in dispersal buffer (0.5% NP-40, 150 mM NaCl, 10 mM Tris-HCl pH 7.4, 2 mM CaCl₂, 22 mM MgCl₂).

In preparation for flow cytometry, cells were resuspended in 300 μl of PBS supplemented with 1% FCS. Cells were then fixed by the drop-wise addition of 900 μl of ice cold 95% ethanol while vortexing and left on ice for 10 minutes. Cells were collected by centrifugation (3,000×g, 5 mins), washed twice in 700 μl of cold PBS before being finally resuspended in PBS. Cells were stained by the addition of 2.5 mg/ml propidium iodide (Sigma) and 0.5 mg/ml RNAase A and analysed on a Beckman-Coulter flow cytometer with WinMDi software.

Cell Lysates, Antibodies and Immunoblot Analysis

Cells were washed once in TEN buffer then harvested by scraping from petri dishes using a rubber policeman, followed by two PBS washes. Cell pellets were collected by centrifugation in microfuge tubes (14,000×g, 1 min), washed twice in PBS, snap frozen and stored at −80° C. Whole-cell lysates were prepared, by resuspending cell pellets at 10⁷ cells/ml in ice-cold lysis buffer (50 mM Hepes pH 7.9, 250 mM KCl, 0.1 mM EDTA, 0.1 mM EGTA, 0.4 mM NaF, 0.4 mM NaVO₄, 10% glycerol, 0.1% NP40, 0.5 nM PMSF, 1 μg/ml leupeptin, 1 mM DTT) for one hour on ice with intermittent vortexing. Lysates were clarified by centrifugation at 14,000×g for 10 minutes at 4° C. and protein concentrations determined by Bradford assay using a BioRad kit. Cell lysates were either snap frozen and stored at −80° C. or used immediately for immunoprecipitations.

Rabbit polyclonal antibodies raised against cyclin E (M-20), cyclin A (C-19), Cdk2 (M-2), cyclin B1 (H-433), E2F4 (C-20), p107 (SD-9X), p27 (C-19), Oct4 (N-19) were from Santa Cruz, anti-peptide polyclonal antibodies, recognizing Cdc2 (#9112) and Y¹⁵ phosphorylated Cdc2 (#9111) were from Cell Signalling Technology, p21 (HJ21) antibodies were from NeoMarkers. The mouse monoclonal raised against β-tubulin (YOL1/34) was from Sera Laboratories. All antibodies were used according to the manufacturers specifications.

Cell lysates were resolved on 8-12% SDS Tris-glycine or Tris-tricine polyacrylamide gels, electrotransferred to nitrocellulose membranes and blocked in PBS containing 0.1% Tween-20 (PBST) and 5% milk powder (Diploma) for at least two hours. Membranes were incubated with primary antibody in PBST supplemented with 1% milk powder for a further two hours, at room temperature or overnight at 4° C. Membranes were then washed 4 times at room temperature for 15 minutes in PBST, then incubated for 1 hour with HRP-conjugated donkey anti-rabbit or sheep anti-mouse secondary antibody (Dako; 1:2,000) in PBST, 1% milk powder. HRP activity was detected with an ECL detection kit (Pierce) on Fuji X-ray film.

Immunoprecipitations, Histone H1 Kinase and Cdc25 Activation Assays.

Protein A Sepharose (PAS) beads (Pharmacia) were stored in BSA (1 mg/ml), 0.05% sodium azide at 4° C. as a 50% slurry. All subsequent procedures were performed at 4° C. unless otherwise specified. Primary antibody was tumbled with PAS beads in lysis buffer for 2 hours; 5 μg IgG was added per 30 μl of PAS beads. Cell lysate (1 mg/ml protein) in a total volume of 300 μl adjusted with lysis buffer, was pre-cleared with 30 μl PAS pre-incubated with 5 μg total rabbit IgG and tumbled for 30 minutes. PAS beads were removed by centrifugation (14,000×g, 10 seconds) and to the pre-cleared supernatant, 30 μPAS beads loaded with primary antibody was added and tumbled for 2 hours. PAS beads were collected by centrifugation (8,000×g, 10 seconds), washed once with kinase lysis buffer (KLB; 50 mM Hepes pH 7.5, 150 mM NaCl, 10% glycerol, 1% Triton X100, 1.5 mM MgCl₂, 1 mM EGTA, 10 μg/μl leupeptin, 1 mM PMSF, 200 μM NaVO₄, 10 mM NaPP, 100 mM NaF, 1 mM DTT), twice with KLB adjusted to 1M NaCl, once with KLB, and three times with 50 mM Hepes pH 7.5, 1 mM DTT. Immune complexes were then resuspended in 20 μl of kinase buffer (50 mM Hepes pH 7.5, 10 mM MgCl₂, 1 mM DTT, 2.5 mM EGTA, 0.1 mM NaOV4, 1 mM NaF) and 5 μl of master mix; 4 μl kinase buffer, 0.5 μl of histone H1 (Boehringer Mannheim) and 0.5 μl of [γ-³²P]-ATP (10 mCi/ml) and incubated at 30° C. for 30 minutes. Kinase reactions were terminated by the addition of an equal volume of 2×SDS load buffer (100 mM Tris-HCl pH 6.8, 20% glycerol, 4% SDS, 200 mM DTT). Phosphorylated histone H1 was resolved using a 12% SDS polyacrylamide gel. [³²P]-labelled histone H1 was quantitated from dried gels using a phosphorimager (Molecular Dynamics).

Protein kinase assays for the characterization of Ro09-3033 were performed as described in (6). Recombinant human Cdc2-cyclin B, Cdk2-cyclin E and Cdk4-cyclin D, expressed from baculovirus expression constructs in insect cells, were kind gifts from Dr Wade Harper (Baylor College of Medicine, Houston, Tex.).

Northern Analysis

Cells were scraped from petri dishes following incubation for 5 minutes in TEN buffer at room temperature. Cells were washed twice and the pellet frozen at −80° C. from which total RNA was extracted using the RNAzol kit (Tel-test). RNA (15 μg) was resolved on 1.5% agarose-formaldehyde gels, transferred to nitrocellulose membranes by capillary action and fixed to the membrane by UV-crosslinking (Stratalinker, Stratagene). Constructs used to generate cDNA fragments for the synthesis of probes for cyclin E, RRMP-2, Cdc2 and B-myb have been described previously (23). Other probe DNA fragments used in this study were as follows; Oct 4, (55); Rex-1 (21); Fgf-5 (19) and mGAP (46). [³²P]-labelled probes were prepared using a Megaprime kit (Amersham) according to the manufacturer's instructions. Following pre-hybridization of filters for at least two hours in UltraHyb (Amersham) at 42° C., denatured probe was added directly to the pre-hybridization mix and allowed to hybridise for 15-18 hours. Membranes were washed twice for 20 minutes in 2×SSC, 0.1% SOS at 42° C. and then twice for 20 minutes in 0.1×SSC at 42° C. Membranes were scanned and hybridisation signals quantitated by scanning on a phosphorimager.

Results

Cell Cycle Structure of Embryonically-Derived Pluripotent Cells

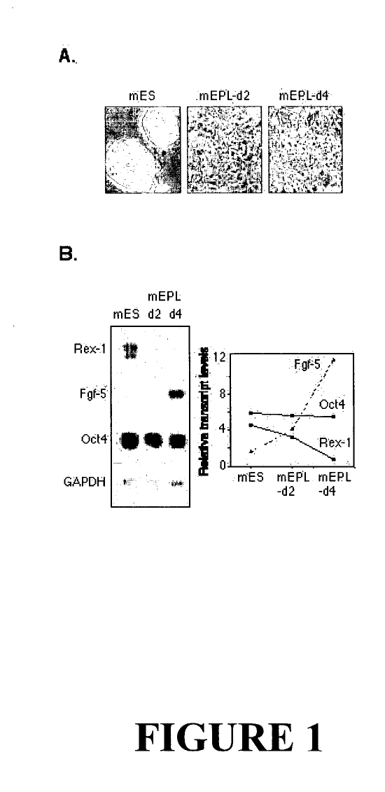

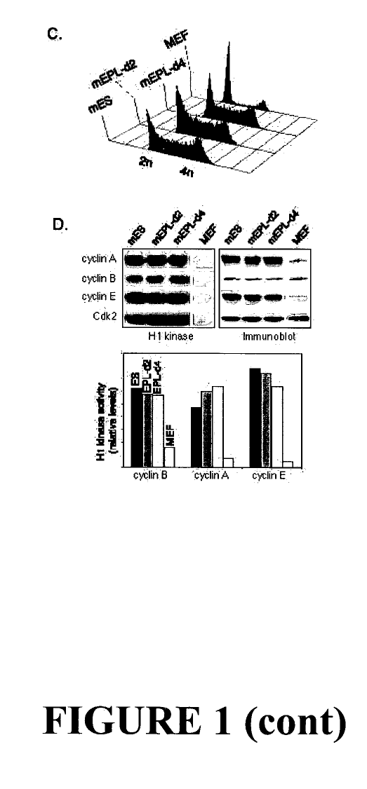

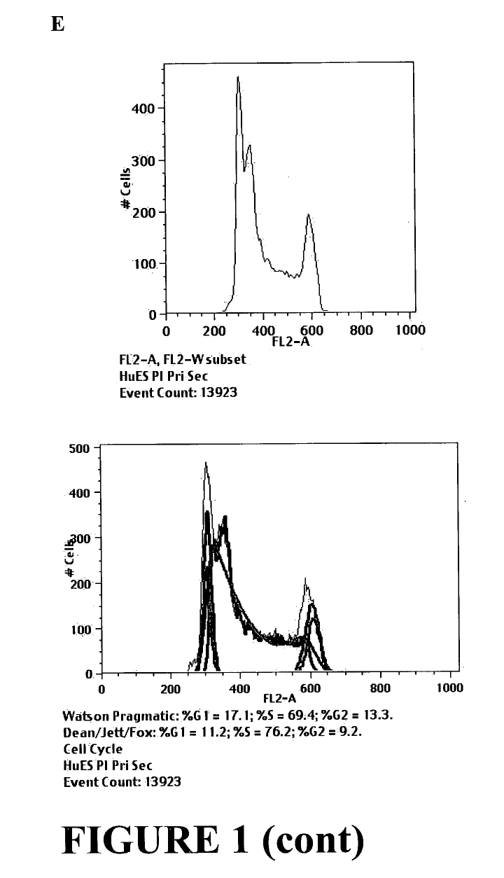

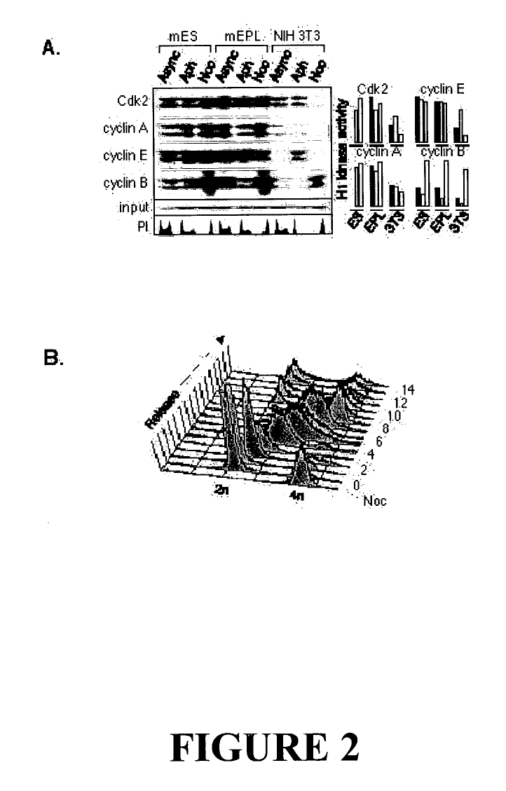

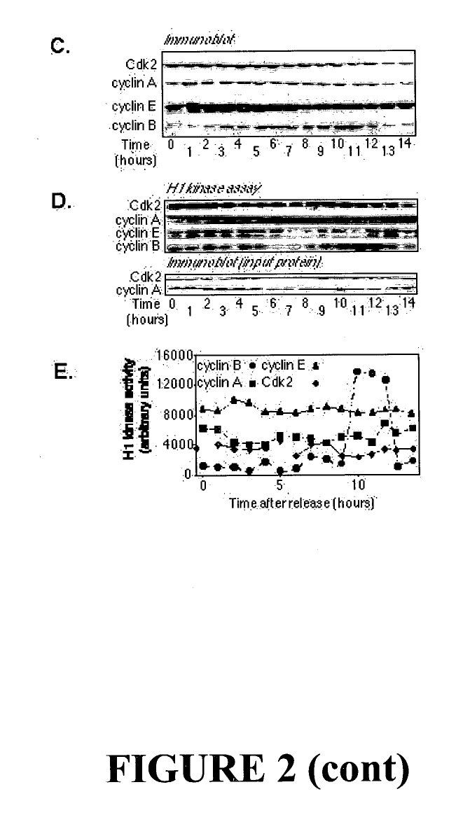

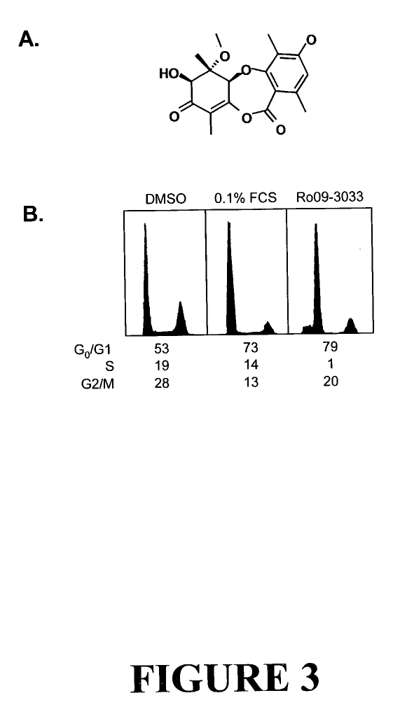

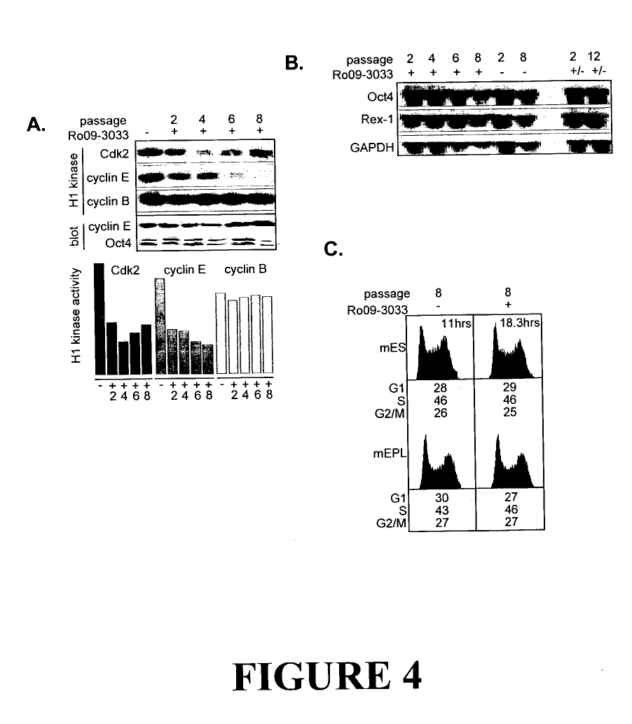

mEPL cells were generated directly from mES cells after 2-4 days culture in MedII conditioned medium and exhibit distinct changes in colony morphology (FIG. 1A) and marker gene expression (FIG. 1B). These cells proceed to differentiate in vitro with characteristics similar to that of early primitive ectoderm (data not shown; 10,11). Flow cytometry analysis showed that the cell cycle structure of mEPL cells was indistinguishable from that of mES cells (FIG. 10). Both cell populations devote significant proportions of time to S-phase and lack fully established gap phases. The mean generation time for D3 mES cells was 12.3 hours and upon conversion to mEPL cells, this was reduced to 8.1 hours. Similar cell cycle profiles and generation times were observed for other mouse ES lines (including E14, CGR8 and MBL5) and corresponding mEPL cells (data not shown). We have also observed similar cell cycle structures for primitive ectoderm surgically dissected from the embryonic epiblast of 6.25dpc mouse embryos (data not shown), consistent with previous observations in the rat (12).

A similar cell cycle structure was observed for human ES cells (FIG. 1E). Cell flow cytometry analysis of human ES cells showed that human ES cells occupy a major proportion of the cell cycle (about 70%) in S phase, and spend minimal time in G1 and G2 phases (about 15% and 10% respectively).

In conclusion, all embryonically-derived pluripotent cells examined exhibit a similar cell cycle structure.

Absence of Cell Cycle Regulated Cdk2 Activity in Murine Pluripotent Cells

To address the underlying mechanisms behind rapid cell proliferation and the unusual cell cycle structure associated with pluripotent cells, we evaluated the activity of several Cdk-associated complexes in asynchronous cell populations. Cdk4,6-cyclin D activities were omitted from this analysis because it has been reported previously that mES cells lack these activities (51) and embryos before 6.0dpc do not express significant levels of cyclin D1, cyclin D2 or cyclin D3 (13).

Murine ES and EPL cells express the somatic cyclin A2 subtype (61), but not the germ line-specific subtype cyclin A1. The predominant form of cyclin E is the E1 subtype.

In comparison to a panel of mouse cell lines including MEFs, NIH 3T3 and Balbc 3T3 fibroblast, mES and mEPL cells display vastly elevated histone H1 kinase activity associated with cyclin A, cyclin B, cyclin E and Cdk2 (FIG. 1D and data not shown). Both cyclin A and cyclin E were expressed at levels well beyond that seen in other primary and transformed mouse cell lines that were assayed, including MEFs and NIH 3T3 fibroblasts. In contrast, Cdk2 levels were comparable to several other cell lines that were evaluated (FIG. 1D and data not shown).

We then asked if these Cdk activities were cell cycle regulated by assaying H1 kinase activity in immunoprecipitates from statically-arrested mES and mEPL cell extracts. Cdk2 activity remained relatively constant between asynchronous, aphidicolin-blocked (predominantly S-phase) and nocodazole-blocked cell populations (G2/M; FIG. 2). Similar assays that evaluated cyclin A and cyclin E-associated Cdk activity also showed little variation between statically-blocked cell populations. These observations are somewhat surprising given the changes in Cdk activities that typically occur during the mammalian cell cycle (14), such as in the case of NIH 3T3 cells (FIG. 2). Levels of cyclin A and cyclin E showed only modest differences (2-3 fold) in the static blocks whereas Cdk2 was constant (data not shown). In contrast, cyclin B-associated activity was significantly higher in nocadozole-blocked cells compared to aphidicolin-blocked cells and was the only cell cycle regulated cyclin-associated HI kinase activity detected in mES and mEPL cells, To confirm that blocked cells retained their pluripotency, Oct4 and alkaline phosphatase status was confirmed and these cells were shown to be capable of in vitro differentiation that was indistinguishable to that of untreated cells (data not shown).

To confirm that Cdk2, cyclin E and cyclin A associated kinase activities are not subject to cell cycle control in mES cells, we employed a synchronization procedure to allow for the evaluation of events over a complete cell division cycle. Conventional synchrony methods were either ineffective or inappropriate for mES cells, in part because of their cell cycle structure, making it necessary to use a nocodazole-aphidicolin synchronization procedure. Cells were initially blocked in nocodazole, then released and trapped at G1/S in the presence of aphidicolin (see Materials and methods). This reproducibly yielded a population of cells (>90%) arrested at the G1-S boundary that could be released into S-phase for synchrony experiments (FIG. 2B). Because the cell cycle of mES cells has a high proportion of S-phase cells and relatively few G1 and G2 cells (see FIG. 1C), it was difficult to follow cells throughout multiple cell cycles. However, the approach allows mES cell cultures to be reliably tracked as a synchronous population for approximately one complete cell division cycle.

Cdk2 protein levels in whole cell lysates showed no obvious periodicity over a complete cell cycle (FIGS. 2C,D) and its histone H1-associated kinase activity showed no obvious signs of periodic regulation (FIG. 2D), consistent with the static block data shown in FIG. 2A. Robust amounts of cyclin E and cyclin A protein were detected by immunoblot analysis at all stages of the cell cycle. Cyclin E levels were however, modestly elevated (1.5-2.0 fold) in S-phase compared to G2/M and G1 cells (FIG. 2C). No cyclic changes in cyclin E or A activities were reproducibly observed in synchronization experiments (FIG. 2C), consistent with our earlier static block results. Cyclin B activity was evaluated in parallel but in contrast to Cdk2, cyclin E and cyclin A activity, was clearly cell cycle-dependent being most active late in the cell cycle, coinciding with G2/M-phase (FIGS. 2C,D). Increased cyclin B-associated kinase activity during G2/M was accompanied by accumulation of cyclin B protein during late-S through to M-phase. Levels of cyclin B collapsed upon re-entry into G1, paralleling the loss of cyclin B-H1 kinase activity (FIGS. 2C,D). These data show that while Cdc2-cyclin B activity shows cell cycle-dependent periodicity, Cdk2 activity is constitutive and that this, in part at least, can be accounted for by elevated levels of cyclins A and E throughout the cell cycle.

Cdk2 Activity Determines Cell Division Rates but not Cell Cycle Structure

Our data suggests that precocious Cdk2 activity in mES and mEPL cells may be a major factor in driving their rapid rate of cell division and in perhaps establishing their unique cell cycle structure. By suppressing Cdk2 activity, we predict that one possible outcome would be to slow the rate of cell division and to increase the relative proportion of time spent in G1, thus establishing a longer gap phase. To test this idea we used a Cdk2-specific inhibitor, Ro09-3033 (6; FIG. 3A), to determine the impact that Cdk2 inhibition has on cell division rates, pluripotency and cell cycle structure. Ro09-3033 is highly selective for Cdk2 (IC₅₀ 20 nM; Table 1), it binds irreversibly to the ATP binding site of Cdk2 (6) and enforces a cell cycle arrest when used in the low micromolar range, in a manner consistent with it targeting Cdk2 in vivo (FIG. 3B).

In mES cells, Ro09-30331 at 2 μM was shown to inhibit immunoprecipitated Cdk2 and cyclin E kinase activities by 50-60% under our standard assay conditions (FIG. 4A). At this concentration and up to 20 μM, Ro09-3033 had no reproducible effect on the activity of other Cdks immunoprecipitated from mES lysates, such as Cdc2-cyclin B (see FIG. 4A and data not shown). Moreover, the inhibitor had no effect on the expression pattern of mES cell marker genes Rex-1 and Oct4 (FIG. 7B), indicating that during the period of inhibitor treatment (up to 8 passages) these cells retained their pluripotent characteristics. The concentration of Ro09-3033 required to produce a uniform cell cycle arrest in mES cells, where the S-phase compartment of cells was depleted, was >5-fold higher than that required for most other cell types tested including Saos2 cells (20 μM, FIG. 6B), NIH 3T3 fibroblasts and MEFs (data not shown), indicating that pluripotent cells are less responsive to this inhibitor. Similar observations were also made with the Cdk2/Cdc2 inhibitor, roscovitine (see below).

Generation times of mES cells in this experiment were 11 hours for untreated and 18.3 hours for inhibitor-treated cells (FIG. 4C) Although a 66% increase in cell cycle time was observed for inhibitor-treated cells this had no major effect on the cell cycle structure, indicating that the increase in cycling time was distributed over all phases. The percentage of cells that incorporated BrdU after a 24 hour labelling period was 98% and 95% for untreated and treated mES cells, respectively, indicating that differences in generation times can not be accounted for by an increase in the proportion of non-cycling cells (data not shown). Moreover, under these conditions Ro09-3033 had no effect on cell viability as judged by PI exclusion and the absence of a sub-2n DNA content. Similar observations were also made for roscovitine which typically acts as a cell cycle specific inhibitor in the low micromolar range (20-30 μM), resulting in the accumulation of cells at G1/S or G2/M (31). In mES cells, roscovitine had no effect on cell division times or cell cycle structure up to concentrations of 140 μM (data not shown). The lowered sensitivity of mES cells to roscovitine and Ro09-3033 may be associated with the precocious activity of Cdk2-cyclin A,E and Cdc2-cyclin B complexes in these cells (see FIG. 1). These results are consistent with the idea that high Cdk activity is a rate-limiting factor underpinning rapid cell division in pluripotent cells of embryonic origin, but this per se is not responsible for their unusual cell cycle structure.

It will be understood that the invention disclosed and defined in the specification extends to all alternative combinations of two or more of the individual features mentioned or evident from the text or drawings. All of these different combinations constitute various alternative aspects of the invention.

It will be understood that the term “comprises” or its grammatical variants as used herein is equivalent to the term “includes” and is not to be taken as excluding the presence of other elements or features.

### REFERENCES

1. Doetschman, T. C., Eistetter, H., Katz, M., Schmidt, W. and Kemler, R. 1985. The in vitro development of blastocyst-derived embryonic stem cell lines: formation of visceral yolk sac, blood islands and myocardium. J. Embryol. Exp. Morph. 87: 27-45.

2. Rathien, J., Lake. J. A., Bettess, M. D., Washington, J. M., Chapman, G. and Rathjen, P. D. 1999. Formation of a primitive ectoderm like cell population, EPL cells, from ES cells in response to biologically derived factors. J. Cell. Sci. 112:601

3. Robertson, E. J. 1987. Teratocarcinomas and embryonic stem cells: A practical approach. p71-112. E. J. Robertson (ed.), IRL Press, Oxford, U.K.

4. Dhingra, U. H., Shirai, H., Takehana, Y., Wovkulich, P. M., Yabukl, N. 1998. Dibenzo-oxazopine and -dioxepine derivatives, their preparation, and their use as antitumor agents. U.S. Pat. No. 5,811,420 (F. Hoffmann-La Roche A. -G., Switz.).

5. Hurford, R. K. Jr., Cobrinik, D., Lee, M. H. and Dyson, N. 1997. pRB and p107/p130 are required for the regulated expression of different sets of E2F target genes. Genes Dev. 11:1447-1463.

6. Scholer, H. R., Dressier, G. R., Balling, R., Rohdewohid, H. and Gruss, P. 1990. Oct-4: a germline-specific transcription factor mapping to the t-complex. EMBO J. 9: 2185-2195.

7. Hosler, B. A., LaRosa, G. J., Grippo, J. P. and Gudas, L. 1989. Expression of Rex-1, a gene containing zinc finger motifs, is rapidly reduced by retinoic acid in F9 teratocarcinoma cells. Mol. Cell. Biol. 9: 5623-5629.

8. Hebert, J. M., Basilico, C., Goldfarb, M., Haub, O. and Martin, G. R. 1990. Isolation of cDNAs encoding four mouse FGF family members and characterization of their expression patterns during embryogenesis. Dev. Biol. 138: 454-463.

9. Rathjen, P. D., Nichols, J., Toth, S., Edwards, D. R., Heath, J. K. and Smith, A. G. 1990. Developmentally programmed induction of differentiating inhibiting activity and the control of stem cell populations. Genes. Dev. 4: 2308-2318.

10. Kranenburg, O., de Groot, R. P., Van der Eb, A. J. and Zantema, A. 1995. Differentiation of P19 EC cells leads to differential modulation of cyclin-dependent kinase activities and to changes in the cell cycle profile. Oncogene. 10: 87-95.

11. Rathjen, J., Lake. J. A., Bettess, M. D., Washington, J. M., Chapman, G. and Rathjen, P. D. 1999. Formation of a primitive ectoderm like cell population, EPL cells, from ES cells in response to biologically derived factors. J. Cell. Sci. 112: 601

12. Krek, W., Ewen, M. E., Shirodkar, S., Arany, Z., Kaelin Jr., W. G. and Livingstone, D. M. 1994. Negative regulation of the growth-promoting transcription factor E2F-1 by a stably bound form of cyclin A-dependent kinase. Cell. 78: 161-172.

13. Spruck, C. H., Kwang-Ai, W. and Reed, S. W. 1999. Deregulated cyclin E induces chromosome instability. Nature. 401: 297-300.

14. Mummery, C. L., van den Brink, C. E. and de Laat, S. W. 1987a. Commitment to differentiation induced by retinoic acid in P19 embryonal carcinoma cells is cell cycle dependent. Dev. Biol. 121: 10-19.

15. Meljer, L. and Kim, S. -H. (1997). Chemical inhibitors of cyclin-dependent kinases. Methods Enzym. 283:113-128.

1. A method of identifying and/or selecting non-differentiating pluripotent or pluripotent-related cells from a mixed cell population including differentiating and non-differentiating pluripotent or pluripotent-related cells, which method includes reducing kinase activity and/or expression in said cells.

2. A method according to claim 1 wherein said kinase activity and/or expression is the result of a cyclin-dependent protein kinase.

3. A method according to claim 2 wherein said cyclin-dependent protein kinase is Cdk2.

4. A method according to claim 1 wherein said kinase activity and/or expression is reduced by introducing a kinase inhibitor into said cells.

5. A method according to claim 4 wherein said kinase inhibitor is a chemical inhibitor.

6. A method according to claim 5 wherein said kinase inhibitor is selected from one or more of the group consisting of Ro09-3033, roscoviline, olomoucine, butyrolactone and flavopiridol.

7. A method according to claim 1 wherein said pluripotent or pluropotent-related cells are cells of embryonic origin.

8. A method according to claim 7 wherein said cells are selected from embryonic stem (ES) cells and early primitive ectoderm-like (EPL) cells.

9. A method according to claim 1 wherein said pluripotent and/or pluripotent-related cells are of mammalian origin.

10. A method according to claim 9 wherein said cells are of murine origin.

11. A method according to claim 9 wherein said cells are of human origin.

12. A method according to claim 1 wherein said pluripotent-related cells are multipotent cells.

13. A method according to claim 12 wherein said multipotent cells are selected from haematopoietic stem cells and neural stem cells.

14. A method of selectively arresting the cell cycle of non-differentiating pluripotent or pluripotent-related cells exhibiting reduced viability in vitro which method includes reducing kinase activity and/or expression in said cells.

15. A method according to claim 14 wherein said kinase activity is the result of Cdk2.

16. A method according to claim 15 wherein said kinase activity is reduced by introducing a chemical kinase inhibitor into said cells.

17. A method according to claim 16 wherein said kinase inhibitor is selected from the group consisting of Ro09-3003, roscovitine, olomoucine, butyrolactone and flavopiridol.

18. A method according to claim 14 wherein said pluripotent or pluripotent-related cells are cells of embryonic origin.

19. A method of modifying the cell cycle of a non-differentiating pluripotent and/or pluripotent-related cell which method includes reducing kinase activity and/or expression in said cell.

20. A method according to claim 19 wherein said modification of the cell cycle results in a slowing of the cell cycle without substantially altering the cell cycle structure.

21. A method according to claim 20 wherein said kinase activity is the result of Cdk2.

22. A method according to claim 21 wherein said kinase activity is reduced by introducing a chemical kinase inhibitor into said cells.

23. A method according to claim 22 wherein said kinase inhibitor is selected from the group consisting of Ro09-3003, roscovitine, olomoucine, butyrolactone and flavopiridol.

24. A method according to claim 20 wherein said pluripotent or pluripotent-related cells are cells of embryonic origin.

25. A method of regulating the mitotic and/or physiological activities and differentiation potential of a pluripotent or pluripotent-related cell which method includes reducing kinase activity and/or expression in said cells.

26. A method according to claim 25 wherein said kinase activity is the result of Cdk2.

27. A method according to claim 26 wherein said kinase activity is reduced by introducing a chemical kinase inhibitor into said cells.

28. A method according to claim 27 wherein said kinase inhibitor is selected from a group consisting of Ro09-3003, roscovitine, olomoucine, butyrolactone and flavopiridol.

29. A method according to claim 25 wherein said pluripotent or pluripotent-related cells are cells of embryonic origin.

30. A method according to claim 29 wherein said pluripotent cells are selected from one or more of the group consisting of epiblast cells, ES cells, EPL cells, primordial germ cells (PGCs) and embryonic carcinoma cells.

31. A method according to claim 25 wherein said pluripotent-related cells are multipotent cells.

32. A method according to claim 31 wherein said multipotent cells are selected from haematopoietic stern cells and neural stem cells.

33. Mammalian pluripotent or pluripotent-related cells whenever prepared according to the method according to claim 1.

34. Human pluripotent or pluripotent-related cells whenever prepared according to the method according to claim 1.

35. Pluripotent or pluripotent-related cells that are competent for genetic manipulation whenever prepared according to the method according to claim 1.

36. Embryos derived from pluripotent or pluripotent-related cells according to claim 33.

37. Animals derived from embryos according to claim 36.

38. Use of pluripotent or pluripotent-related cells according to claim 33 in therapies selected from any one of the group consisting of cell therapy, gene therapy, cancer therapy, regeneration and/or development of organs or appendages or limbs, production and/or use in pharamaceuticals and/or diagnostics, xenotransplantation, genetic modification of animals and nuclear transfer.

39. Use of pluripotent or pluripotent-related cells according to claim 33 in drug delivery.
